(19) United States
(12) Patent Application Publication
(10) Pub. No.: US 2021/0378844 A1
(43) Pub. Date: Dec. 9, 2021

(54) PROSTHETIC SYSTEM COMPRISING PROGRAMMED MAGNETS

(71) Applicant: Medtronic, Inc., Minneapolis, MN (US)

(72) Inventors: William C. Harding, Chandler, AZ (US); Shayla O'Brien, Phoenix, AZ (US); Michael R. Arnold, Gilbert, AZ (US); Seth O'Brien, Phoenix, AZ (US)

(21) Appl. No.: 17/337,990

(22) Filed: Jun. 3, 2021

Related U.S. Application Data
(60) Provisional application No. 63/034,746, filed on Jun. 4, 2020.

(51) Int. Cl. A61F 2/80; A61F 2/78; A61F 2/68

(52) U.S. Cl. CPC A61F 2/80; A61F 2/7812; A61F 2002/6863; A61F 2002/7818; A61F 2/68

(57) ABSTRACT

In one example of the present disclosure, a system may be configured with a prosthetic liner configured to be worn over a residual limb of a user where the prosthetic liner comprises a first one or more programmed magnets. The system may be further configured with a prosthesis comprising a prosthetic socket configured to receive the residual limb and the prosthetic liner where the prosthetic socket comprises a second one or more programmed magnets. The first one or more programmed magnets are magnetically attracted to and repelled by corresponding ones of the second one or more programmed magnets when the prosthetic liner is received within the prosthetic socket.

This application claims the benefit of U.S. Provisional Application Ser. No. 63/034,746, filed Jun. 4, 2020, the entire content of which is incorporated herein by reference.

### TECHNICAL FIELD

This disclosure relates to features incorporated into or with a prosthesis to provide suspension and enhance patient comfort.

### BACKGROUND

Most modern artificial limbs may be suspended (e.g., attached) to a residual limb (e.g., stump) of the amputee by sleeves, locking devices, belts, cuffs, or by suction. The residual limb either directly fits into a socket on the prosthesis, or—more commonly—a liner may he used and then is fixed to the socket either by vacuum (e.g., suction sockets) or a pin lock. Liners are soft, and consequently may create a far better fit than hard sockets. Silicone or other elastomeric gel liners may be obtained in standard sizes, mostly with a circular (round) cross section, but for any other residual limb shape, custom liners may be made.

The socket may be custom made to fit the residual limb and to distribute the forces of the artificial limb across the area of the residual limb (i.e., rather than just one small spot), which helps reduce wear on the residual limb. The custom socket may be created by taking a plaster cast of the residual limb or, more commonly, of the liner worn over the residual limb, and then making a mold from the plaster cast. Newer methods include laser-guided measuring which may be input directly to a computer allowing for a more sophisticated design.

Amputees experience considerable discomfort and pain associated with prosthetic limbs. One problem with the residual limb and socket attachment is that a bad fit will reduce the area of contact between the residual limb and socket or liner and increase pockets between the residual limb skin and socket or liner. Pressure then is higher at points of contact, which may be painful. Air pockets may allow sweat to accumulate and may soften the skin. Accumulated sweat is a frequent cause for itchy skin rashes, which over time, may lead to breakdown of the skin. For this reason, current prostheses represent a potential for open wounds, which may lead to infection. Additionally, improper residual limb and socket attachment fit may similarly lead to increased material wear and tear with associated cost.

### SUMMARY

In general, the present disclosure describes examples and techniques in which programmed magnets are included in prostheses. In some examples, the programmed magnets exhibit spring-like magnetism, and may be referred to herein as spring type programmed magnets. In some examples, programmed magnets are included as part of a liner or sleeve configured to be worn by a user, e.g., a patient, over a residual limb. In such examples, programmed magnets are also included as part of a prosthetic device (e.g., an arm, leg, hand or foot) that includes a socket configured to receive the liner. The programmed magnets may allow for limited to no contact between the patient and the prosthetic device, which may mitigate issues with imperfect residual limb and socket attachment and increase patient comfort.

The programmed magnets may be arranged in magnetic pairs, with the liner and the prosthetic device including respective ones of each pair of programmed magnets. The programmed magnets may be configured, e.g., designed or programmed, to simulate the functionality of a spring with compression and tension forces when brought close enough together to magnetically interact. In such examples, as the programmed magnetic pairs get closer to one another than a predefined distance, they will repel each other (e.g., like a spring when compressed together), and will repel with stronger forces if pushed closer together. In such examples, when pulled apart from one another beyond the predefined distance, the programmed magnets will be attracted to one another and become more difficult to separate (e.g., like a spring when you pull two ends of the spring apart); thus, the simulated impression of a spring between the magnets. In this manner, one or more pairs of spring type programmed magnets may be configured to maintain a predefined distance between each other. The programmed magnets may also act as a vibration isolation between the patient and the prosthetic by filtering out vibration over a range of frequencies and mass.

In one example of the present disclosure, a system may be configured with a prosthetic liner configured to be worn over a residual limb of a user where the prosthetic liner comprises a first one or more programmed magnets. The system may be further configured with a prosthesis comprising a prosthetic socket configured to receive the residual limb and the prosthetic liner where the prosthetic socket comprises a second one or more programmed magnets. The first one or more programmed magnets are magnetically attracted to and repelled by corresponding ones of the second one or more programmed magnets when the prosthetic liner is received within the prosthetic socket.

In another example of the present disclosure, a prosthetic system may be configured with a prosthetic liner configured to be worn over a residual limb of a user. The prosthetic system may be further configured with a prosthetic socket configured to receive the residual limb and the liner. A plurality of programmed magnets where a first one or more of the plurality of programmed magnets is coupled to the prosthetic liner and have a spring-like magnetic response when placed proximate to a second one or more of programmed magnets coupled to the prosthetic socket.

In another example of the present disclosure a method of manufacturing a prosthetic system may involve forming a prosthetic liner with a first one or more programmed magnets where the prosthetic liner is configured to be worn over a residual limb of a user. The method may further involve forming a prosthesis with a second one or more programmed magnets where the prosthesis comprises prosthetic socket configured to receive the prosthetic liner. The method may further involve configuring the programmed magnets such that the first one or more programmed magnets are magnetically attracted to and repelled by corresponding ones of the second one or more programmed magnets when the prosthetic liner is received within the prosthetic socket.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram of an example lower-extremity prosthetic system including programmed magnets.

FIG. 2 is a diagram of another example prosthetic system with programmed magnets.

FIG. 3 is a diagram of programmed magnets with magnetic lines of flux illustrating the spring-like magnetism between the programmed magnets.

FIG. 4 is a diagram illustrating an example interface between a liner and socket of a prosthetic system, including first and second sets of programmed magnets.

FIG. 5 is a flow diagram of a method of manufacturing a prosthetic system in accordance with examples of the present disclosure.

### DETAILED DESCRIPTION

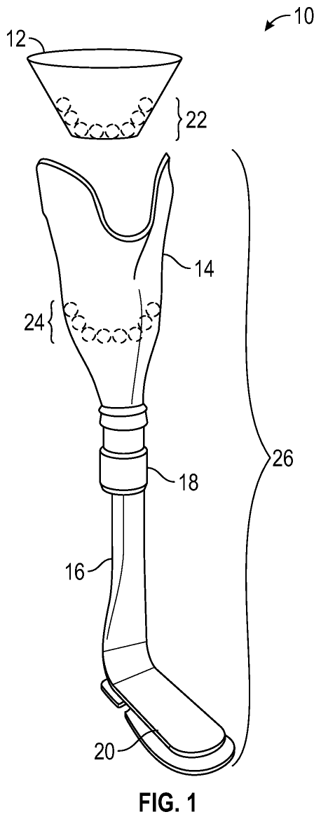

FIG. 1 is a conceptual diagram of an example lower-extremity prosthetic system 10 including programmed magnets. While prosthetic system 10 may be shown as a lower-extremity prosthetic system, the examples and techniques of the present disclosure may be applied to an upper-extremity prosthetic system as well. Prosthetic system 10 may include any prosthetic device, such as a hand, foot, lower leg, lower arm, leg, arm, ear, finger(s), or toe(s), as the examples of the present disclosure may be extended to any part of the human body which may be substituted with a prosthesis.

In the example illustrated by FIG. 1, lower-extremity prosthetic system 10 (hereinafter “prosthetic system 10”) includes a prosthetic liner 12 and prosthesis 26 that includes, a socket 14, a shank 16, a pylon 18, and a foot 20. Prosthesis 26 may also be referred to as a prosthetic device. The examples of this disclosure may be implemented in prosthetic systems in which the prosthesis has a different configuration, e.g., includes fewer components or additional components. Prosthetic liner 12 may also be referred to herein as a liner.

Liner 12 may have a first set of one or more programmed magnets 22. programmed magnets 22 may be embedded, formed, or otherwise included in the liner material, e.g., even with, recessed below, or protruding from an outer surface of liner 12, or attached or otherwise located on the outer surface of the liner 12. Further, a second set of one or more spring-like magnets 24 may be similarly included in a material of socket 14 or located on a surface of socket 14 that may be adjacent to, e.g., in contact with, the outer surface of the liner, when received in the socket.

When brought into sufficient proximity with a predefined orientation relative to one another, programmed magnets 22 and 24 work, e.g., in pairs, to simulate the functionality of a spring with forces resisting changes from a predefined separation distance between programmed magnets 22 and programmed magnets 24. In this manner, programmed magnets 22 and 24 act as an attachment mechanism between liner 12 and socket 14, resisting axial movement of programmed magnets 22 away from programmed magnets 24 and, thus, of liner 12 out of socket 14. In this manner, programmed magnets 22 and 24 also act to reduce contact and transmission of forces between socket 14 and the residual limb (not shown in FIG. 1) within liner 12.

Liner 12 may be made of silicone, silicone gel, urethane or other elastomeric materials and/or several fabrics such as wool, cotton, or various synthetic materials. A prosthetic sock may be worn over liner 12 in order to allow for volume fluctuation.

Prosthetic system 10 may be lightweight. In some examples, several components of prosthetic system 10 may be made from plastic. Socket 14 may be made from polypropylene, or lightweight metals, such as titanium and aluminum, or alloys thereof, in some examples. In some examples, socket 14 may be made from wood (such as maple, hickory basswood, willow, poplar, and linden) and rubber. In some examples, components of system 10, such as socket 14, may be made of composite materials such as carbon fiber or fiberglass.

Socket 14 serves as an interface between the residual limb, also referred to as the residuum, and foot 20, ideally allowing comfortable weight-bearing, movement control and proprioception. These sockets or “interfaces” may be made more comfortable by lining them with a softer, compressible plastic, gel, or foam material providing padding for the bone prominences. Longer limbs may require the use of a locking roll-on type liner or more complex harnessing to help augment suspension.

Shank 16 is the structural component of the prosthesis that would most closely represent the anatomical shin (tibia and fibula bones), and may include prosthetic components such as the foot, pylon and/or structural connectors. The type of connectors used between socket 14 and the knee/foot (based upon amputation level) determines whether prosthesis 26 may be modular (endoskeletal) or not. Modular means the components and alignment (e.g., angles, length, rotation, etc. of foot 20 with respect to socket 14) may be changed after fitting.

Pylon 18 may be the member which provides the connection between the socket 14 and prosthetic foot 20. Pylon 18 may be coupled to foot 20, and to socket 14, which receives the residual limb covered by liner 12. Pylon 18 may be the portion of prosthesis 26 transferring weight between socket 14 and prosthetic foot 20. Pylon 18 may be configured to reduce the shock transmitted to the residual limb each time the heel strikes the ground. This may be particularly important for people who participate in high-impact activities like running or other sports. The spring-like action of a dynamic pylon also helps move the person forward, compressing upon heel strike and then releasing when the toe lifts off the ground. Some pylons 18 have torque-absorption allowing for internal and external rotation of the foot. The amount of resistance may be adjusted depending on the component. Torque absorption may be an important feature for people who play golf or tennis or even dancing. Pylon 18 may be made from steel, titanium, aluminum, or other lightweight metals. Carbon fiber may be used to form a lightweight pylon 18, in some examples.

Foot 20 provides contact to the ground, shock absorption and stability during stance. Additionally, foot 20 influences gait biomechanics by its shape and stiffness. The foot selection and dynamic characteristics are matched to the user's size, weight and activity level, and play a significant role in the effort to normalizing the users gait pattern. There may be several different types of feet with greatly varying characteristics related to durability and biomechanics.

Foot 20 may be made from urethane foam with a wooden inner keel construction. Other materials commonly used may be composites such as carbon fiber or fiberglass, plastics such as polyethylene, polypropylene, acrylics, and polyurethane. Physical appearance of the prosthetic limb may be important to the amputee. Some endoskeletal prostheses (e.g., pylons 18) may be covered with a soft polyurethane foam cover designed to match the shape of the patient's sound limb. This foam cover may be then covered with a sock or artificial skin painted to match the patient's skin color.

Prosthetic system 10 may be an example of an artificially replaced limb for an amputation occurring between the hip and knee. Lower-extremity amputations may be estimated worldwide between 2.0-5.9 per 10,000 people. Estimates of birth prevalence rates of congenital limb deficiency range between 3.5-7.1 cases per 10,000 births. The two main subcategories of lower extremity prosthetic devices may be trans-tibial (e.g., any amputation transecting the tibia bone or a congenital anomaly resulting in a tibial deficiency), and trans-femoral (e.g., any amputation transecting the femur bone or a congenital anomaly resulting in a femoral deficiency). In the prosthetic field, a trans-tibial prosthetic leg may be often referred to as a “BK” or below-the-knee prosthesis, while the trans-femoral prosthetic leg may be often referred to as an “AK” or above-the-knee prosthesis.

Other, less prevalent lower extremity cases include the following: (1) hip disarticulations, e.g., when an amputation or congenital deficiency occurs at the hip joint; (2) knee disarticulations, e.g., an amputation or deficiency through the knee joint; and (3) Symes, through the ankle joint while preserving the heel pad.

Socket issues, such as discomfort and skin breakdown, may be rated among the most important issues faced by amputees. Individuals with amputation resultant from diabetes and other dysvascular conditions, may experience considerable skin breakdown which creates the potential for open wounds and subsequent infection. Additionally, some systems have high material wear and tear associated.

One problem with residual limb and socket attachment may be that a bad fit will reduce the area of contact between the residual limb and socket 14 or liner 12 and increase pockets between residual limb skin and socket 14 or liner 12. Pressure then may be higher and painful. Air pockets may allow sweat to accumulate and may soften the skin. Ultimately, this may be a frequent cause for itchy skin rashes. Over time, this may lead to breakdown of the skin.

Prosthetic system 10 enhances the abilities of a patient to not only move and function normally, but to also perform these movements comfortably, for greater lengths of time and without pain or injury. Prosthetic system 10 addresses issues associated with patient comfort, some of which is discussed above. Examples and techniques of the present disclosure increase patient comfort and reduce the probability of other issues associated with imperfect socket-residual limb fit through programmed magnets 22 and 24 working in pairs to simulate the functionality of a spring with compression and tension forces. programmed magnets 22 and 24 may create and work to maintain a gap between prosthetic liner 12 and socket 14, while also working to keep socket 14 secured to the residual limb. This gap assists in patient comfort by reducing pressure.

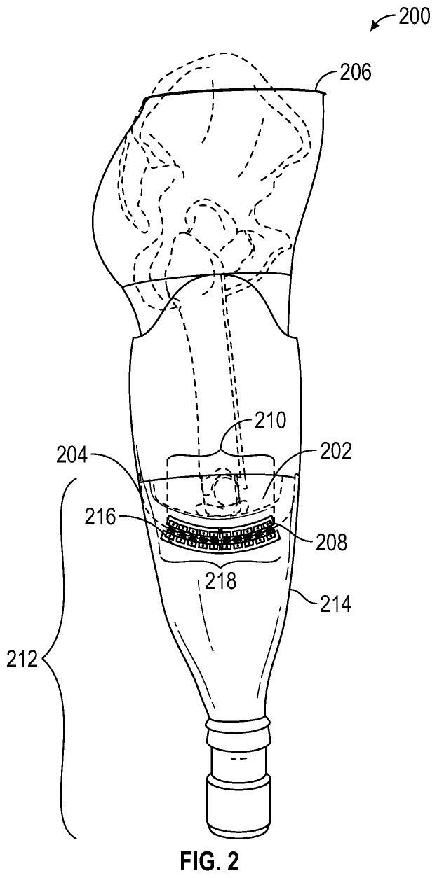

FIG. 2 is a diagram of an example prosthetic system with programmed magnets. A prosthetic system 200, may have a prosthetic liner 204 configured to be worn over a residual limb 202 of a user 206. Prosthetic liner 204 may have a first set 208 of one or more programmed magnets 210.

Prosthetic 212 may have a second set 216 one or more programmed magnets 218. First set 208 of one or more programmed magnets 210 may be magnetically attracted to and repelled by corresponding second set 216 of one or more programmed magnets 218 when prosthetic liner 204 may be received within prosthetic socket 214.

First set 208 and second set 210 of programmed magnets may be illustrated in FIGS. 1 and 2 as respective rows of programmed magnets. However, one or both sets 208, 210 of programmed magnets may include two-dimensional arrays of programmed magnets. Furthermore, the sets 208, 210 of programmed magnets may be positioned substantially within respective planes, or arranged in corresponding non-planer configurations, e.g., substantially conforming to corresponding shapes of the outer and inner surfaces of liner 204 and socket 214, respectively.

When liner 204 is received in socket 214, and absent any forces applied to the liner and/or the socket, e.g., to bring them together or pull them apart, first set 208 having first one or more individual programmed magnets 210 may rest a predefined distance apart from second set 216 of second one or more individual programmed magnets 218. This predefined distance may be determined by the configuration (e.g., referred to as programming) of programmed magnets 210 and/or 218. The distance will be less with a small repulsive magnetic force and greater with a larger repulsive magnetic force between respective ones of the first 208 and second set of programmed magnets 216. The level of magnetic force may be configured based upon the weight of user 206. If user 206 may be a child or smaller adult (e.g., under 100 lbs.), then a small magnetic force may provide enough distance and spring to the programmed magnets 210 and/or 218. If user 206 may be a medium build adult (e.g., between 100 and 200 lbs.), then programmed magnets 210 and/or 218 may have a mid-range magnetism to support the additional weight of user 206. Further, if user 206 may be heavier, greater than for example 200 lbs., then magnets 210 and/or 218 may have a strong magnetism to support the weight of user 206 and provide the spring like effect to provide comfort and vibration dampening and a predefined distance between sets 208 and 216.

The predefined distance between sets 208 and 216 provide many benefits to user 206 as well as prosthetic 212. programmed magnetic sets 208 and 216 provide impact resistance between prosthetic liner 204 and socket 214, which may reduce pain for user 206. The impact resistance also reduces wear and tear on prosthetic liner 204 and socket 214. Wear and tear may be a large concern for user 206 as many prosthetics may be very expensive. The reduced wear and tear may help extend the life of prosthetic 212. The reduced impact may improve the health of residual limb 202. The reduced pain and comfort during use of prosthetic 212 may assist in improving the prosthetic user's state of mind. The reduction in pain may make user 206 use prosthetic 212 more, do more activities and improve social connectivity.

Prosthetic system 200 may address many issues associated with user comfort and use of prosthetics. For example, programmed magnets 210 and/or 218 may act to attach prosthetic 212 to residual limb 202 and or maintain orientation of the prosthetic with the residual limb. programmed magnets 210 and/or 218 work in pairs to simulate the functionality of a spring between residual limb 202 and prosthetic 212 with compression and tension forces. As discussed, first set of programmed magnets 208 and second set of programmed magnets 216 work to maintain a predefined distance apart from each other, repelling when pushed together and attracting when pulled apart.

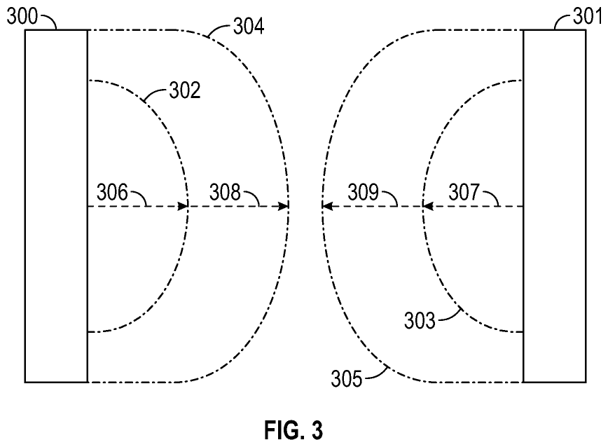

FIG. 3 is a diagram of programmed magnets 300 and 301 with magnetic lines of flux 302, 304, 303 and 305 illustrating the spring-like magnetism between the programmed magnets 300 and 301. programmed magnets 300 and 301 may have magnetic lines of flux represented in FIG. 3 as a repel line of flux 302 and 303 and an attraction line of flux 304 and 305.

The magnetic attraction force may be the weakest at the farthest end of vectors 308 and 309 away from programmed magnets 300 and 301, respectively. As programmed magnets 300 and 301 may be moved closer to one another, attraction lines of flux 304 and 305 will begin to intersect. At this point a weak magnetic attraction force will begin to act on each of programmed magnets 300 and 301 and they will be drawn toward one another.

As programmed magnets 300 and 301 are magnetically pulled together by the magnetic attraction the magnetic attraction will grow stronger the further attraction lines of flux 304 and 305 move along vectors 308 and 309 toward repel lines of flux 302 and 303. As the attraction lines of flux 304 and 305 begin to engage and cross over the repel lines of flux 302 and 303, programmed magnets 300 and 301 will begin to repel each other weakly. The magnetic strength of repulsion may be the weakest at the end of vectors 306 and 307 furthest away from programmed magnets 300 and 301 respectively.

If there were a force, such as the body weight of a prosthetic user, on either of programmed magnet 300 and/or 301 that may cause programmed magnets 300 and 301 to be pushed closer together, the magnetic strength of repulsion may grow greater the closer programmed magnets 300 and 301 got to one another. For example, the strength of spring effect and attraction will be configured based on the patient's physiological properties and their use case (e.g., running, climbing, walking, hiking, etc.). That said, the spring and attraction will not be a constant across all patients.

Equilibrium, where programmed magnets 300 and 301 will balance their attraction and their repulsion, may be associated with the predefined distance between the programmed magnets. This predefined distance may be mathematically represented by:

Sum of the repulsion vectors [306 & 307])+(Distance of strongest attraction vector [308 or 309])=predefined distance.

This may be the equilibrium state where, without any forces acting on programmed magnets 300 and 301, programmed magnets 300 and 301 will remain repelled from one another but attracted just enough to remain at a predefined distance apart from one another.

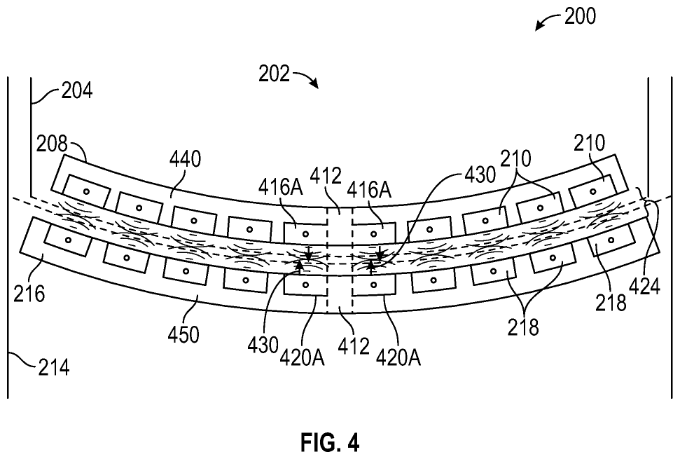

FIG. 4 is a diagram illustrating an example interface between a liner 204 and prosthetic device 212 of a prosthetic system 200, including first 208 and second sets 216 of programmed magnets 210 and 218. A prosthetic system 200 may have a first set 208 of one or more programmed magnets 210 that may be within, woven into or reside on a prosthetic liner 204 to be worn over a residual limb 202 of a user 206. A second set 216 of one or more programmed magnets 218 that may be woven into, within, molded part of or reside on a prosthetic socket 214 that may receive prosthetic liner 204. First set 208 of one or more programmed magnets 210 may be magnetically attracted to and repelled by corresponding ones of second set 216 of one or more programmed magnets 218 when prosthetic liner 204 is received within prosthetic socket 214.

Prosthetic liner 204 may have an orientation channel 412. Orientation channel 412 may be configured to receive an orientation pin, if needed. When prosthetic liner 204 is received within prosthetic socket 214 an orientation pin may be received within orientation channel 412 and maintain an orientation of first set 208 of one or more programmed magnets 210 with the corresponding ones of second set 216 of programmed magnets 218. Prosthetic liner 204 may be received within prosthetic socket 214 and an orientation channel 412 may be parallel to at least one pair of programmed magnets 210, 218 in one of first set 208 or second set 216 programmed magnets 210, 218. Orientation channel 412 may also be parallel to magnetic vectors of flux 430 emanating from programmed magnets 416A and 420A, which may be the programmed magnets closest to the orientation channel 412. In some examples, there may be multiple orientation channels with multiple corresponding orientation pins extending along liner 204 and socket 414. An orientation pin may extend from orientation channel 412 formed in liner 204. In other examples one or more orientation pins may extend from liner 204, and one or more orientation channels 412 may be formed in socket 214.

First set 208 of programmed magnets 210 will rest a predefined distance 424 apart from second set 216 of programmed magnets 218. As discussed above, first set 208 of programmed magnets 210 will repel second set 216 of programmed magnets 218 when pushed closer together. First set 208 of programmed magnets 210 will be attracted to second set 216 of programmed magnets 218 when pulled apart.

Second set of programmed magnets 216 may be located proximate a surface of prosthetic 426 that defines prosthetic socket 414. First set 402 of one or more programmed magnets 404 may have several programmed magnets 408. Second set 208 of one or more programmed magnets 210 may have several programmed magnets 210. Positions of first set 208 of programmed magnets 210 may define a first non-planar shape and positions of second set 216 of magnets 218 may define a second non-planar shape corresponding to the first non-planar shape.

Membrane 440 may be of a flexible material, such as silicon, a polymer or even a cloth material such as cotton. Membrane 440 may be flexible to adapt to prosthetic liner 204 which may be made of a flexible material to better fit the user's residual limb 202. Membrane 440 may reside within, sowed into or reside on prosthetic liner 204. Membrane 450 may be made of a rigid material as the socket 414 may be made of a rigid material. However, membrane 450 may be made of most any material, such as metal, wood, carbon fiber, or a polymer. Membrane 450 may be built into, reside on or placed within prosthetic socket 214.

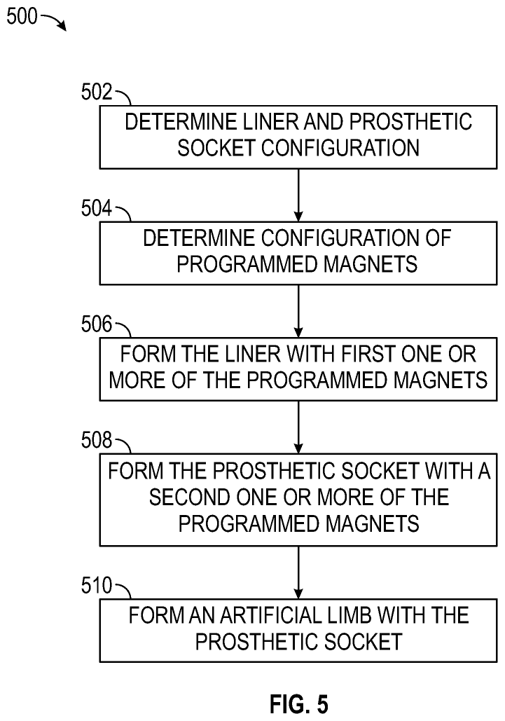

FIG. 5 is a flow diagram of an example method of manufacturing a prosthetic system 500 in accordance with examples of the present disclosure. A method of manufacturing a prosthetic 500 may have the following processes performed in any manner unless specifically stated otherwise. Prosthetic limbs may not be mass-produced to be sold in stores. Like the way dentures or eyeglasses may be procured, prosthetic limbs may first be prescribed by a medical doctor, usually after consultation with the amputee, a prosthetist, and a physical therapist. The patient then visits the prosthetist to be fitted with a limb. Although some parts—the socket, for instance—may be custom-made, many parts (e.g., feet, pylons) may be manufactured in a factory, sent to the prosthetist, and assembled at the prosthetist's facility in accordance with the patient's needs. At a few facilities, the limbs may be custom made from start to finish.

Accuracy and attention to detail may be important in the manufacture of prosthetic limbs, because the goal may be to have a limb that comes as close as possible to being as comfortable and useful as a natural one. Before work on the fabrication of the limb may be begun, the prosthetist evaluates the amputee and takes measurements of the residual limb as well as an impression (cast) or digital reading of the residual limb. This may be made of plaster of Paris, because it dries fast and yields a detailed impression. From the plaster cast, a positive model—an exact duplicate—of the stump may be created. The prosthetist will then modify the shape of the positive model to facilitate proper fit and weight bearing of the prosthetic socket.

A sheet of clear thermoplastic may be heated in a large oven and then vacuum-formed around the positive model. In this process, the heated sheet may be simply laid over the top of the model and placed under vacuum, collapsing the sheet around the mold and forcing it into the exact shape of the mold. This thermoplastic sheet may now be the test socket; it may be transparent so that the prosthetist may check the fit.

The prosthetist works with the patient to ensure that the test socket fits properly. In the case of a missing leg, the patient walks while wearing the test socket, and the prosthetist studies the gait. Because the material from which the test socket may be made may be thermoplastic, it may be reheated to make minor adjustments in shape. Adjustments may be made to optimize fit, comfort and gait characteristics.

A permanent socket may then be formed out of plastics (e.g. polypropylene) or composite materials (e.g. laminated carbon fiber or fiberglass). It may be common for the stump to shrink after surgery, stabilizing approximately 1-3 years later. Thus, the socket may be replaced at that time, and thereafter when anatomical changes necessitate a change.

Plastic pieces—including soft-foam pieces used as liners or padding—may be made in the usual plastic forming methods (502). These include vacuum-forming, injection molding—forcing molten plastic into a mold and letting it cool—and extruding, in which the plastic may be pulled through a shaped die.

A plurality of programmed magnets having spring-like magnetism may be programed and created (504). The programmed magnets may be formed in most any method including 3D printing and may consider factors such as the patient's (user's) weight to properly program the desired magnetic repulsion and attraction. The programmed magnets incorporate correlated patterns of magnets with alternating polarity, designed to achieve the behavior described above in FIG. 3 and deliver stronger local force. By varying the magnetic fields and strengths, different mechanical behaviors may be controlled.

programmed magnet pairs may be programmed to attract or repel with a prescribed force and engagement distance, or, to attract or repel at a certain spatial orientation. programmed magnets may be programmed to interact only with other magnetic structures that have been coded to respond. programmed magnets may even be programmed to attract and repel at the same time. Compared to conventional magnets, programmed magnets provide stronger holding force to the target and stronger shear resistance. The programmable behavior may be achieved by creating multipole structures comprising multiple magnetic elements of varying size, location, orientation, and saturation. The sizes of these spring-magnetic elements may range from 1 mm to 4 mm. By overlapping these magnetic elements, a very intricate magnetic field may be produced.

Programmed magnets may be programmed, by varying the polarity and/or field strengths of each source of the arrays of magnetic sources that make up each structure. The resulting magnetic structures may be one-dimensional, two-dimensional, and three-dimensional if produced using an electromagnetic array.

Correlated magnetic structures may be developed from ferrites, rare-earth materials (e.g. Neodymium magnet, Samarium—cobalt magnet, neodymium iron boron), ceramics, and electromagnets alike, and the correlation effects may be scalable from very large permanent magnets to nanometer-scale devices. Multipole magnetic devices may be constructed from discrete permanent magnets, or by exposing heated magnetizable material to a magnetic field.

The magnets may then be formed together with the liner (506). The socket may also be formed with the magnets (508).

Pylons that may be made of titanium or aluminum may be die-cast; in this process, liquid metal may be forced into a steel die of the proper shape. The wooden pieces may be planed, sawed, and drilled. The various components may be put together in a variety of ways, using bolts, adhesives, and laminating, to name a few.

The entire limb may be assembled by the prosthetist using such tools as a torque wrench and screwdriver to bolt the prosthetic device together (510). After this, the prosthetist again fits the permanent socket to the patient, this time with the completed custom-made limb attached. Final adjustments may then be made.

The following is a non-limiting list of examples that are in accordance with one or more techniques of this disclosure.

Example 1A. A system comprising: a prosthetic liner configured to be worn over a residual limb of a user, wherein the prosthetic liner comprises a first one or more programmed magnets; and a prosthesis comprising a prosthetic socket configured to receive the residual limb and the prosthetic liner, wherein the prosthetic socket comprises a second one or more programmed magnets, wherein the first one or more programmed magnets are magnetically attracted to and repelled by corresponding ones of the second one or more programmed magnets when the prosthetic liner is received within the prosthetic socket.

Example 2A. The system of example 1A, wherein the prosthetic socket and the prosthetic liner further comprise an orientation channel configured to receive an orientation pin.

Example 3A. The system of example 2A, wherein, when the prosthetic liner is received within the prosthetic socket and the orientation pin is received within the orientation channel, the orientation pin and orientation channel maintain a predefined orientation of the first one or more programmed magnets with the corresponding ones of the second programmed magnets.

Example 4A. The system of any one of examples 2A or 3A, wherein, when the prosthetic liner is received within the prosthetic socket, the orientation channel extends parallel to a vector of the magnetic fields between at least one pair of programmed magnets comprising one of the first one or more programmed magnets and one of the second one or more programmed magnets.

Example 5A. The system of any one of examples 1A, 2A, 3A, or 4A, wherein the programmed magnets are 3D printed.

Example 6A. The system of any one of examples 1A, 2A, 3A, 4A or 5A, wherein the corresponding ones of the first one or more programmed magnets and the second one or more programmed magnets will rest a predefined distance apart from one another when the prosthetic liner is received within the prosthetic socket.

Example 7A. The system of example 6A, wherein the corresponding ones of the first one or more programmed magnets and the second one or more programmed magnets will repel each other when pushed closer together than the predefined distance.

Example 8A. The system of example 7A, wherein the corresponding ones of the first one or more programmed magnets and the second one or more programmed magnets will be attracted to each other when pulled further apart than the predefined distance.

Example 9A. The system of any one of examples 1A, 2A, 3A, 4A, 5A, 6A, 7A or 8A, wherein the second one or more programmed magnets are located proximate a surface of the prosthetic configured to contact the prosthetic liner when received within the socket.

Example 10A. The system of any one of examples 1A, 2A, 3A, 4A, 5A, 6A, 7A, 8A, or 9A, wherein the first one or more programmed magnets comprise a first plurality of programmed magnets, and the second one or more programmed magnets comprise a second plurality of programmed magnets.

Example 11A. The system of example 10A, wherein each of the first plurality of programmed magnets corresponds to a respective one of the second plurality of programmed magnets.

Example 12A. The system of any one or examples 10A or 11A, wherein positions of the first plurality of magnets define a first non-planar shape, and positions of the second plurality of magnets define a second non-planar shape corresponding to the first non-planar shape.

Example 1B. A prosthetic system comprising: a prosthetic liner configured to be worn over a residual limb of a user; a prosthetic socket configured to receive the residual limb and the liner; and a plurality of programmed magnets, wherein a first one or more of the plurality of programmed magnets coupled to the prosthetic liner have a spring-like magnetic response when placed proximate to a second one or more of programmed magnets coupled to the prosthetic socket.

Example 2B. The system of example 1B, further comprising an orientation channel configured to align the prosthetic liner and the prosthetic socket when worn by the user.

Example 1C. A method of manufacturing a prosthetic system comprising: forming a prosthetic liner with a first one or more programmed magnets, wherein the prosthetic liner is configured to be worn over a residual limb of a user; forming a prosthetic with a second one or more programmed magnets, wherein the prosthetic comprises prosthetic socket configured to receive the prosthetic liner; and configuring the programmed magnets such that the first one or more programmed magnets are magnetically attracted to and repelled by corresponding ones of the second one or more programmed magnets when the prosthetic liner is received within the prosthetic socket.

Example 2C. The method of example 1C, wherein forming the prosthetic comprises forming an orientation channel.

Example 3C. The method of any one of examples 1C or 2C, wherein configuring the programmed magnets comprises configuring the programmed magnets such that the corresponding ones of the first one or more programmed magnets and the second one or more programmed magnets will rest a predefined distance apart from one another when the prosthetic liner is received within the prosthetic socket.

Example 4C. The method of any one of examples 1C, 2C, or 3C, further comprising printing, with a 3D printer, the plurality of magnets.

Example 5C. The method of any one of examples 1C, 2C, 3C, or 4C, wherein configuring the programmed magnets comprises configuring a respective strength of spring-like magnetism for each pair of one of the one or more first magnets with the corresponding one of the second one or more magnets.

Example 6C. The method of any one of examples 1C, 2C, 3C, 4C, or 5C, wherein configuring the programmed magnets comprises configuring the magnets based on at least one of: corresponding shapes of the residual limb, the liner and the prosthetic socket, painful or sensitive areas on the residual limb, a size of the user, a weight of the user, or a physical activity profile of the user.

Various examples have been described herein. Any combination of the described operations or functions may be contemplated. These and other examples may be within the scope of the following claims.

What is claimed is:

1. A system comprising:
a prosthetic liner configured to be worn over a residual limb of a user, wherein the prosthetic liner comprises a first one or more programmed magnets; and
a prosthesis comprising a prosthetic socket configured to receive the residual limb and the prosthetic liner, wherein the prosthetic socket comprises a second one or more programmed magnets,
wherein the first one or more programmed magnets are magnetically attracted to and repelled by corresponding ones of the second one or more programmed magnets when the prosthetic liner is received within the prosthetic socket.

2. The system of claim 1, wherein the prosthetic socket and the prosthetic liner further comprise an orientation channel configured to receive an orientation pin.

3. The system of claim 2, wherein, when the prosthetic liner is received within the prosthetic socket and the orientation pin is received within the orientation channel, the orientation pin and orientation channel maintain a predefined orientation of the first one or more programmed magnets with the corresponding ones of the second programmed magnets.

4. The system of claim 2, wherein, when the prosthetic liner is received within the prosthetic socket, the orientation channel extends parallel to a vector of the magnetic fields between at least one pair of programmed magnets comprising one of the first one or more programmed magnets and one of the second one or more programmed magnets.

5. The system of claim 1, wherein the programmed magnets are 3D printed.

6. The system of claim 1, wherein the corresponding ones of the first one or more programmed magnets and the second one or more programmed magnets will rest a predefined distance apart from one another when the prosthetic liner is received within the prosthetic socket.

7. The system of claim 6, wherein the corresponding ones of the first one or more programmed magnets and the second one or more programmed magnets will repel each other when pushed closer together than the predefined distance.

8. The system of claim 7, wherein the corresponding ones of the first one or more programmed magnets and the second one or more programmed magnets will be attracted to each other when pulled further apart than the predefined distance.

9. The system of claim 1, wherein the second one or more programmed magnets are located proximate a surface of the prosthetic configured to contact the prosthetic liner when received within the socket.

10. The system of claim 1, wherein the first one or more programmed magnets comprise a first plurality of programmed magnets, and the second one or more programmed magnets comprise a second plurality of programmed magnets.

11. The system of claim 10, wherein each of the first plurality of programmed magnets corresponds to a respective one of the second plurality of programmed magnets.

12. The system of claim 10, wherein positions of the first plurality of magnets define a first non-planar shape, and positions of the second plurality of magnets define a second non-planar shape corresponding to the first non-planar shape.

13. A prosthetic system comprising:
a prosthetic liner configured to be worn over a residual limb of a user;
a prosthetic socket configured to receive the residual limb and the liner; and
a plurality of programmed magnets, wherein a first one or more of the plurality of programmed magnets coupled to the prosthetic liner have a spring-like magnetic response when placed proximate to a second one or more of programmed magnets coupled to the prosthetic socket.

14. The system of claim 13, further comprising an orientation channel configured to align the prosthetic liner and the prosthetic socket when worn by the user.

15. A method of manufacturing a prosthetic system comprising:
forming a prosthetic liner with a first one or more programmed magnets, wherein the prosthetic liner is configured to be worn over a residual limb of a user;
forming a prosthetic with a second one or more programmed magnets, wherein the prosthetic comprises prosthetic socket configured to receive the prosthetic liner; and
configuring the programmed magnets such that the first one or more programmed magnets are magnetically attracted to and repelled by corresponding ones of the second one or more programmed magnets when the prosthetic liner is received within the prosthetic socket.

16. The method of claim 15, wherein forming the prosthetic comprises forming an orientation channel.

17. The method of claim 15, wherein configuring the programmed magnets comprises configuring the programmed magnets such that the corresponding ones of the first one or more programmed magnets and the second one or more programmed magnets will rest a predefined distance apart from one another when the prosthetic liner is received within the prosthetic socket.

18. The method of claim 15, further comprising printing, with a 3D printer, the plurality of magnets.

19. The method of claim 15, wherein configuring the programmed magnets comprises configuring a respective strength of spring-like magnetism for each pair of one of the one or more first magnets with the corresponding one of the second one or more magnets.

20. The method of claim 15, wherein configuring the programmed magnets comprises configuring the magnets based on at least one of: corresponding shapes of the residual limb, the liner and the prosthetic socket, painful or sensitive areas on the residual limb, a size of the user, a weight of the user, or a physical activity profile of the user.
